(19) United States
(12) Patent Application Publication
(10) Pub. No.: US 2008/0071273 A1
(43) Pub. Date: Mar. 20, 2008

(54) Dynamic Pedicle Screw System

(76) Inventors: David T. Hawkes, Pleasant Grove, UT (US); Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/856,469

(22) Filed: Sep. 17, 2007

Related U.S. Application Data
(60) Provisional application No. 60/844,981, filed on Sep. 15, 2006.

(51) Int. Cl. A61B 17/58; A61B 17/56

(52) U.S. Cl. 606/61; 606/103; 606/73

(57) ABSTRACT

A system for stabilizing at least one spinal motion segment includes a fastener including an anchoring portion and a coupling portion, and a longitudinal support member coupled to the fastener, wherein a portion of the system is formed from a super-elastic material.

### RELATED APPLICATIONS

The present application claims the benefit under 35 U.S.C. § 119(e) of U.S. Provisional Patent Application No. 60/844,981 filed Sep. 15, 2006 titled “Dynamic Pedicle Screw System,” which application is incorporated herein by reference in its entirety.

### TECHNICAL FIELD

The present exemplary system and method relates to medical devices. More particularly, the present exemplary system and method relates to dynamic orthopedic implantable devices.

### BACKGROUND

Traumatic, inflammatory, metabolic, synovial, neoplastic and degenerative disorders of the spine can produce debilitating pain that can affect a spinal motion segment's ability to properly function. The specific location or source of spinal pain is most often an affected intervertebral disc or facet joint. Often, a disorder in one location or spinal component can lead to eventual deterioration or disorder, and ultimately, pain in the other.

Spine fusion (arthrodesis) is a procedure in which two or more adjacent vertebral bodies are fused together. It is one of the most common approaches to alleviating various types of spinal pain, particularly pain associated with one or more affected intervertebral discs. While spine fusion generally helps to eliminate certain types of pain, it has been shown to decrease function by limiting the range of motion for patients in flexion, extension, rotation and lateral bending. Furthermore, the fusion creates increased stresses on adjacent non-fused motion segments and accelerated degeneration of the motion segments. Additionally, pseudarthrosis (resulting from an incomplete or ineffective fusion) may not provide the expected pain-relief for the patient. Also, the device(s) used for fusion, whether artificial or biological, may migrate out of the fusion site creating significant new problems for the patient.

Various technologies and approaches have been developed to treat spinal pain without fusion in order to maintain or recreate the natural biomechanics of the spine. To this end, significant efforts are being made in the use of implantable artificial intervertebral discs. Artificial discs are intended to restore articulation between vertebral bodies so as to recreate the full range of motion normally allowed by the elastic properties of the natural disc. Unfortunately, the currently available artificial discs do not adequately address all of the mechanics of motion for the spinal column.

It has been found that the facet joints can also be a significant source of spinal disorders and debilitating pain. For example, a patient may suffer from arthritic facet joints, severe facet joint tropism, otherwise deformed facet joints, facet joint injuries, etc. These disorders lead to spinal stenosis, degenerative spondylolithesis, and/or isthmic spondylotlisthesis, pinching the nerves that extend between the affected vertebrae.

Current interventions for the treatment of facet joint disorders have not been found to provide completely successful results. Facetectomy (removal of the facet joints) may provide some pain relief; but as the facet joints help to support axial, torsional, and shear loads that act on the spinal column in addition to providing a sliding articulation and mechanism for load transmission, their removal inhibits natural spinal function. Laminectomy (removal of the lamina, including the spinal arch and the spinous process) may also provide pain relief associated with facet joint disorders; however, the spine is made less stable and subject to hypermobility. Problems with the facet joints can also complicate treatments associated with other portions of the spine. In fact, contraindications for disc replacement include arthritic facet joints, absent facet joints, severe facet joint tropism, or otherwise deformed facet joints due to the inability of the artificial disc (when used with compromised or missing facet joints) to properly restore the natural biomechanics of the spinal motion segment.

Recently, surgical-based technologies, referred to as dynamic posterior stabilization, have been developed to address spinal pain resulting from more than one disorder, when more than one structure of the spine have been compromised. An objective of such technologies is to provide the support of fusion-based implants while maximizing the natural biomechanics of the spine. Dynamic posterior stabilization systems typically fall into one of two general categories: posterior pedicle screw-based systems and interspinous spacers.

One shortcoming of traditional posterior pedicle screw-based stabilization systems is that forces created by the systems are often translated to the anchored pedicle screws. Often, the skeletally mature patients have a relatively brittle bone structure that cannot withstand the transfer of these forces; resulting in failure of the anchoring system.

### SUMMARY

In one of many possible exemplary embodiments, the present system provides for stabilizing at least one spinal motion segment including a fastener having an anchoring portion and a coupling portion, and a longitudinal support member coupled to the fastener, wherein a portion of the system is formed from a super-elastic material.

In yet another of many possible exemplary embodiments, a method for generating a dynamic support structure, includes inserting at least one fastener into a desired orthopedic location, and coupling a longitudinal support member to the at least one fastener, wherein either the at least one fastener or the longitudinal support member includes a super-elastic material.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIGS. 1A and 1B illustrate a dynamic stabilization system including a superelastic rod in an assembled view and an exploded view, respectively, according to one exemplary embodiment.

FIG. 2 is a stress-strain diagram illustrating the characteristics of a super-elastic material, according to one exemplary embodiment.

FIG. 3 is a side view of a dynamic pedicle screw configuration, according to one exemplary embodiment.

FIG. 4 is a side view of a dynamic pedicle screw configuration including a screw head, according to one exemplary embodiment.

FIGS. 5A and 5B are an exploded view and a partial cross sectional assembled view, respectively of a press-on dynamic stabilization system, according to one exemplary embodiment.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar, but not necessarily identical, elements.

### DETAILED DESCRIPTION

The present exemplary systems and methods provide an implantable connection system that can be used to create a dynamic stabilization system. According to one exemplary embodiment of the present system and method, a portion of the stabilization construct includes a shape memory or superelastic metal configured to flex without becoming permanently deformed. Particularly, according to one exemplary embodiment, the ability to flex reduces the transfer of motion forces to the anchoring device, thereby preventing failure of the anchoring device in skeletally mature patients or other patients having brittle skeletal systems.

As used herein, and in the appended claims, the term “super elastic material” shall be interpreted broadly as including any metal, metal alloy, plastic, or composite material exhibiting shape memory. Particularly, according to one exemplary embodiment, a super elastic or shape memory material is a material, typically a metallic alloy such ad Nitinol (NiTi), that, after an apparent applied deformation, has the ability to recover to its original shape upon heating or a reduction in stress due to a reversible solid-state phase transformation.

In the following description, for purposes of explanation, numerous specific details are set forth in order to provide a thorough understanding of the present system and method for providing an implantable dynamic stabilization system. It will be apparent, however, to one skilled in the art, that the present method may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

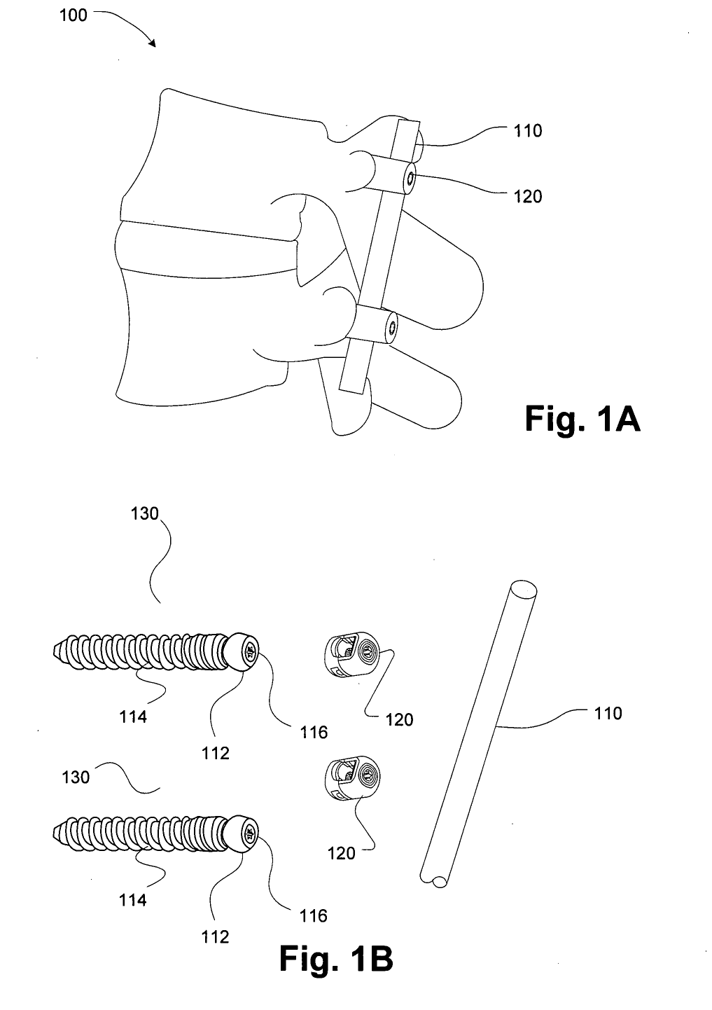

FIGS. 1A and 1B illustrate assembled and exploded views, respectively, of an exemplary dynamic stabilization system that can incorporate a super elastic material into the stabilization system, according to one exemplary embodiment. As illustrated in the assembled stabilization system of FIG. 1A, an anchoring system (100) is provided including a longitudinal rod (110) and a tulip assembly (120). As further illustrated in FIG. 1B, the tulip assembly (120) also includes a pedicle screw (130) configured to be coupled to a vertebrae thereby providing the vertebral connection for the exemplary anchoring system.

As illustrated in the exemplary embodiment illustrated in FIG. 1B, the anchoring system (100) includes a longitudinal rod (110). According to one exemplary embodiment, the longitudinal rod (110) is a cylindrical support configured to engage the tulip assemblies (120) and at least partially secure the relative positions of the tulip assemblies.

The tulip assemblies (120) are configured to fix (e.g., lock) the longitudinal rod (110) to the pedicle screw (130) at a desired angle either before or after inserting and/or capturing the rod. The present exemplary tulip assembly (120) may be configured to initially lock the longitudinal rod (110) to the pedicle screw (130) to reduce and/or prevent any translational and/or rotational movement of the tulip assembly relative to the pedicle screw. The ability to initially lock the tulip assembly to the pedicle screw may facilitate the surgeon in performing compression and/or distraction of various spinal and/or bone sections. While an exemplary tulip assembly (120) is illustrated in FIGS. 1A and 1B, any number of available tulip assemblies (120) may be used with the present exemplary anchoring system (100) including, but in no way limited to, tulip assemblies illustrated in U.S. Pat. App. Nos. 20060161153, 20060161152, and 20060155278.

As illustrated in FIG. 1B, the exemplary pedicle screw (130) includes a pedicle screw (130) having a head or a head portion (112). According to the exemplary embodiment illustrated in FIG. 1B, the pedicle screw (130) includes both an elongated, threaded portion (114) and a head portion (115). Although pedicle screws (130) are generally known in the art, the head portions (112) may be of varying configurations depending on what type of tulip assembly is to be coupled to the pedicle screw (130). The head portion (112) of the present exemplary pedicle screw (130) includes a driving feature (116) and a maximum diameter portion. The driving feature (116) of the present exemplary pedicle screw (130) permits the screw to be inserted into a pedicle bone and/or other bone. According to one exemplary embodiment, the pedicle bone is a part of a vertebra that connects the lamina with a vertebral body. Additionally, according to the present exemplary embodiment, the driving feature (116) can be used to adjust the pedicle screw (130) prior to or after the tulip assembly is coupled to the pedicle screw (130). In the illustrated embodiment, the head portion (112) of the pedicle screw (130) is coupled to the threaded portion (114) and includes a generally spherical surface with a truncated or flat top surface.

In one exemplary embodiment, the pedicle screw (130) is cannulated, which means a channel (not shown) extends axially through the entire length of the pedicle screw (130). The channel (not shown) allows the pedicle screw (130) to be maneuvered over and receive a Kirschner wire, commonly referred to as a K-wire. The K-wire is typically pre-positioned using imaging techniques, for example, fluoroscopy imaging, and then used to provide precise placement of the pedicle screw (130). While the pedicle screw (130) illustrated in FIG. 1B includes a number of components, numerous variations may be made including, but in no way limited to, varying the type of driving feature (116), varying the head shape, varying materials, varying dimensions, varying the location of the threads, including necking features, and the like.

As mentioned previously, the present exemplary system is configured to provide an implantable connection system that can be used to create a dynamic stabilization system. Particularly, according to one exemplary embodiment, either the top portion of the exemplary pedicle screw (130) or at least a portion of the longitudinal rod itself (110) is formed of a shape memory alloy or superelastic metal. By forming a portion of the present exemplary anchoring system (100) of a shape memory alloy such as a superelastic metal, forces created by the systems and translated to the anchored pedicle screws is greatly reduced. Consequently, failure of the anchoring system (100) is also greatly reduced.

According to a first exemplary embodiment, the longitudinal rod (110) is formed of a shape memory alloy or superelastic metal. As shown in FIG. 1A, traditional pedicle screws and tulip assemblies (120) may be inserted into a patient's spine. After insertion, a longitudinal rod (110) formed of the shape memory alloy or superelastic metal may be coupled to the tulip assemblies (120). As a result of using the shape memory alloy or superelastic metal as the longitudinal rod (110), twisting and bending are allowed while providing support to the construct and reducing transfer of forces to the anchoring mechanisms. Specifically, according to the present exemplary embodiment, when a twisting force is imparted on the exemplary anchoring system (100) including a longitudinal rod (110) formed of a superelastic metal, at least a portion of the resulting force is transmitted to the deformation of the longitudinal rod (110), rather than to the thread portion of the pedicle screw (130).

According to one exemplary embodiment of the present exemplary anchoring system (100), the super-elastic material used to form the one or more exemplary flexible sections may be a shape memory alloy (SMA). Super-elasticity is a unique property of SMA. If an SMA is deformed at a temperature slightly above its transition temperature, it quickly returns to its original shape. This super-elastic effect is caused by the stress-induced formation of at least some martensite above its normal temperature. Consequently, when an object composed of SMA has been formed above its transition temperature and a stress is induced to the resulting object, the martensite reverts immediately to undeformed austenite as soon as the stress is removed.

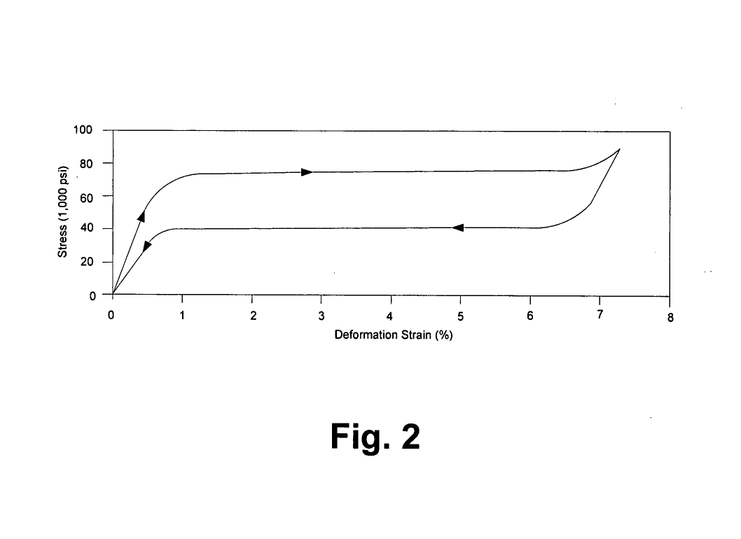

FIG. 2 is a stress/strain diagram illustrating the stress/strain properties of a super-elastic material used for the exemplary flexible sections of the present exemplary anchoring system (100), according to one exemplary embodiment. As shown, an initial increase in deformation strain creates great stresses in the material, followed by a stress plateau with the continued introduction of strain. As the strain is reduced, the stress reduces sharply and again plateaus, providing a substantially constant level of stress which is lower than the initial level of constant stress. This property of the super-elastic material allows the flexible sections of the present exemplary anchoring system (100) to be preloaded with compressive forces prior to or once inserted into the system, thereby providing support to the anchoring system construct.

According to one exemplary embodiment, the super-elastic material used to form the flexible sections may include, but is in no way limited to a shape memory alloy of nickel and titanium commonly referred to as Nitinol. According to this exemplary embodiment, one advantage of the Nitinol being that it can flex (withstand higher stresses) much more than standard materials such as titanium, without becoming permanently deformed. According to one exemplary embodiment, the diameter of the flexible section(s) will be varied and sized to produce the desired flexibility and spring constant.

Additionally, Nitinol may be selected as the material used to produce the flexible section(s), according to one exemplary embodiment, because Nitinol wire provides a low constant force at human body temperature. Particularly, the transition temperature of Nitinol wire is such that Nitinol wires generate force at the standard human body temperature of about 37° C. (98.6° F.).

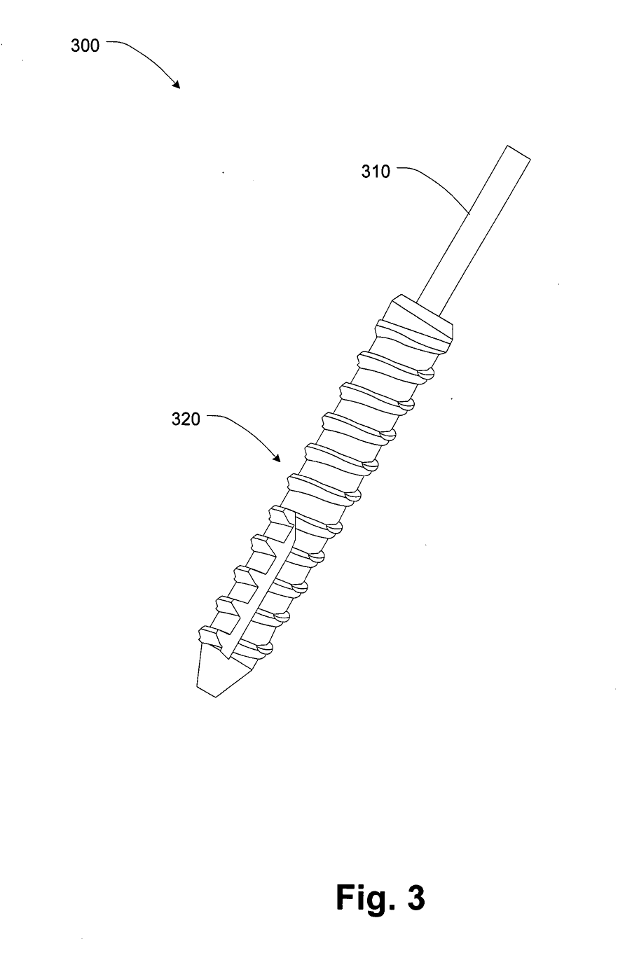

While the above mentioned exemplary anchoring system (100) is described as having the longitudinal rod (110) formed of a shape memory alloy or superelastic metal, other portions of the exemplary anchoring system (100) may be formed of a shape memory alloy or superelastic metal. Particularly, according to one exemplary embodiment, the exemplary anchoring system (100) may include a dynamic pedicle screw system (300) including at least a portion of the dynamic pedicle screw system (300) being formed of a superelastic material. As illustrated in FIG. 3, the exemplary dynamic pedicle screw system (300) includes an anchoring portion (320) and a flexing portion (310). According to one exemplary embodiment, the anchoring portion (320) may include, but is in no way limited to a threaded portion. As shown, the anchoring portion (320) may include a self-tapping screw system to facilitate insertion thereof.

Continuing with FIG. 3, the exemplary pedicle screw system (300) includes a flexing portion (310) configured to provide twisting and bending in a resulting stabilization construct or anchoring system (100). Particularly, according to one exemplary embodiment, the present exemplary dynamic pedicle screw system (300) is configured to be inserted in a plurality of pedicles. One or more stabilization rod(s) (110) may then be coupled to the flexing portion (310) of the exemplary dynamic pedicle screw system via a tulip or other connector member. As a result, the inserted pedicle screw systems (300) will provide a proper spacing for the resulting construct, while allowing twisting and bending in the construct. When bending and twisting do occur, the resulting forces are at least partially absorbed by the flexing portion (310), resulting in a bending of the flexing portion. Consequently, the entirety of the resulting forces is not transferred to the anchoring portion (320) of the pedicle screw system (100).

According to one exemplary embodiment, any number of driving features may be formed on the exemplary pedicle screw system (100). Particularly, according to one exemplary embodiment, a driving feature (not shown) may be formed between the flexing portion (110) and the anchoring portion (120) to allow for the exemplary pedicle screw system (100) to be driven into a desired spinal location.

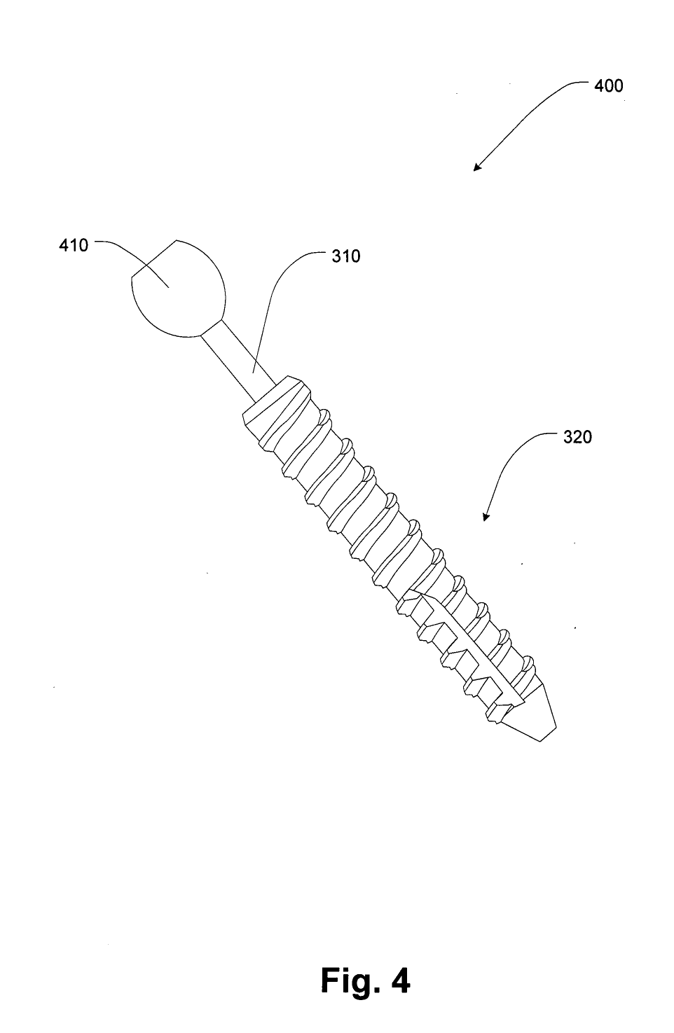

FIG. 4 illustrates an alternative pedicle screw system (400), according to one exemplary embodiment. As illustrated in FIG. 4, the alternative exemplary pedicle screw system (400) includes an anchoring portion (320) and a flexing portion (310) as previously described. However, in contrast to the exemplary pedicle screw system (300) embodiment illustrated in FIG. 3, the alternative pedicle screw system (400) illustrated in FIG. 4 includes a screw head (410) or other driving feature. According to this exemplary embodiment, the screw head (410) may be used to drive the anchoring portion (320) into a desired orthopedic location. After insertion, a tulip or other connector member may be coupled to the screw head (410).

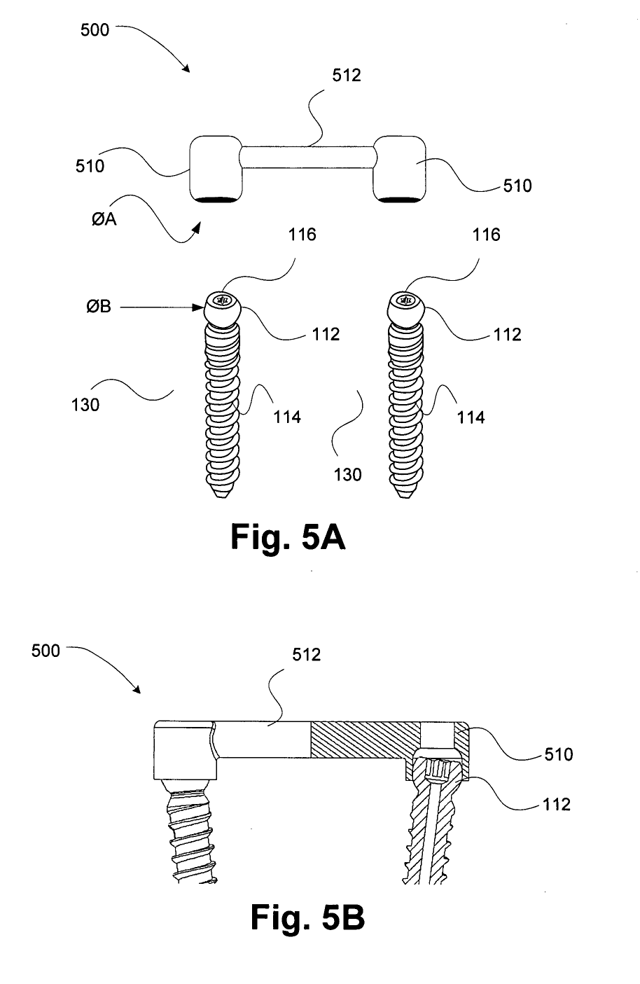

FIGS. 5A and 5B illustrate another exemplary pedicle screw configuration (500) that may be used to provide a dynamic stabilization system, according to one exemplary embodiment. As illustrated in FIG. 5A, traditional pedicle screws (110) may be used with the exemplary configuration. Alternatively, the afore-mentioned pedicle screws described above with reference to FIGS. 3 and 4 could also be used. In place of a separate tulip and rod assembly, as is described above, the exemplary embodiment illustrated in FIGS. 5A and 5B includes a rod-coupling element including a rod or connection member (512) extending from or spanning between one or more screw head receptacles (510). According to one exemplary embodiment, the screw head receptacles (510) are formed having an internal diameter (ØA) for receiving a spherical screw head of a second diameter (ØB), where ØA is smaller than ØB, such that when said components are pressed together, they create a press fit or an interference fit between the components to prevent motion.

To engage the screw head receptacle (510) to the screw head (112), an instrument would engage the underside of the screw head (112) and apply a load to the top of the screw head receptacle (510) to press the components together. Disassembly is achieved by pulling up on the rod-coupling element (512) while driving a ram through the center of the screw head receptacle (510) to push out the screw head (112). As mentioned previously, the screw head receptacle (510) and the rod-coupling element (512) may all be made of a superelastic material such as Nitinol. According to this exemplary embodiment, every element of the configuration may be made of a superelastic material, providing the ability to design in any degree of flexure in the configuration (500).

As mentioned previously, a shape memory alloy or superelastic metal is used to allow twisting and bending in the illustrated systems. The advantage of the shape memory alloy or superelastic metal being that it can flex (withstand higher stresses) much more than titanium or other traditional materials, without becoming permanently deformed. According to the present exemplary system, the diameter of the flexible sections will be sized to produce the desired flexibility as determined by any number of factors including, but in no way limited to, damage to the patient, age of the patient, orthopedic health of the patient, and the like.

A number of preferred embodiments of the present exemplary system and method have been described and are illustrated in the accompanying Figures. Nevertheless, it will be understood that various modifications may be made without departing from the spirit and scope of the present exemplary systems and methods. For example, while the exemplary implementations have been described and shown using screws to anchor into bony structures, the scope of the present exemplary system and methods is not so limited. Any means of anchoring can be used, such as a cam, screw, staple, nail, pin, or hook.

The preceding description has been presented only to illustrate and describe embodiments of the present exemplary systems and methods. It is not intended to be exhaustive or to limit the systems and methods to any precise form disclosed. Many modifications and variations are possible in light of the above teaching. It is intended that the scope of the invention be defined by the following claims.

What is claimed is:

1. A system for stabilizing at least one spinal motion segment, comprising:
a fastener including an anchoring portion and a coupling portion; and
a longitudinal support member coupled to said fastener;
wherein at least a portion of said system is formed from a super-elastic material.

2. The system of claim 1, wherein said super-elastic material comprises a shape memory alloy.

3. The system of claim 2, wherein said shape memory alloy comprises Nitinol.

4. The system of claim 1, wherein said portion of said system formed from a super-elastic material comprises at least a portion of said longitudinal support member.

5. The system of claim 1, wherein said portion of said system formed from a super-elastic material comprises at least a portion of said fastener.

6. The system of claim 5, wherein said coupling portion of said fastener comprises a super-elastic material.

7. The system of claim 6, further comprising a driving head coupled to said super-elastic material.

8. The system of claim 1, wherein a diameter of said super-elastic material is designed to produce a desired flexibility.

9. The system of claim 1, further comprising a tulip assembly configured to couple said fastener assembly to said longitudinal support member.

10. The system of claim 9, wherein:
said tulip assembly and said longitudinal support member are permanently coupled to form a single structure;
wherein said single structure is entirely formed of said super-elastic material.

11. A system for stabilizing at least one spinal motion segment, comprising:
a fastener including an anchoring portion and a coupling portion; and
a longitudinal support member coupled to said fastener;
wherein at least a portion of said system is formed of Nitinol.

12. The system of claim 11, wherein said portion of said system formed of Nitinol comprises a selected portion of said longitudinal support member.

13. The system of claim 11, wherein said portion of said system formed of Nitinol comprises said longitudinal support member.

14. The system of claim 11, wherein said portion of said system formed from a super-elastic material comprises at least a portion of said fastener.

15. The system of claim 14, wherein said coupling portion of said fastener comprises a super-elastic material.

16. The system of claim 15, further comprising a driving head coupled to said super-elastic material.

17. The system of claim 11, wherein a diameter of said super-elastic material is designed to produce a desired flexibility.

18. The system of claim 11, further comprising a tulip assembly configured to couple said fastener assembly to said longitudinal support member.

19. The system of claim 18, wherein:
said tulip assembly and said longitudinal support member are permanently coupled to form a single structure;
wherein said single structure is entirely formed of said super-elastic material.

20. A method for generating a dynamic support structure, comprising:
inserting at least one fastener into a desired orthopedic location; and
coupling a longitudinal support member to said at least one fastener;
wherein either said at least one fastener or said longitudinal support member includes a super-elastic material.

21. The method of claim 20, further comprising sizing said super-elastic material to provide a desired flexibility.

22. The method of claim 20, further comprising:
coupling a tulip assembly to said at least one fastener; and
coupling said longitudinal support member to said tulip assembly.
